(19) United States
(12) Patent Application Publication
(10) Pub. No.: US 2020/0129524 A1
(43) Pub. Date: Apr. 30, 2020

(54) PERSONALIZED CONTRACEPTIVE FORMULATIONS

(71) Applicant: Agile Therapeutics, Inc., Princeton, NJ (US)

(72) Inventors: Agis KYDONIEUS, Kendall Park, NJ (US); Elizabeth I.O. Garner, Bryn Mawr, PA (US); Joseph A. Chiodo, III, Wall, NJ (US); Joseph A. D'Urso, Princeton, NJ (US)

(73) Assignee: Agile Therapeutics, Inc., Princeton, NJ (US)

(21) Appl. No.: 16/494,123

(22) PCT Filed: Mar. 13, 2018
(86) PCT No.: PCT/US2018/022247

§ 371 Date: Sep. 13, 2019

Related U.S. Application Data
(60) Provisional application No. 62/471,640, filed on Mar. 15, 2017.

(51) Int. Cl. A61K 31/567; A61K 31/573; A61K 31/565; A61P 15/18; A61K 9/70

(52) U.S. Cl. CPC A61K 31/567; A61K 31/573; A61K 9/7023; A61P 15/18; A61K 31/565

(57) ABSTRACT

Formulations, kits and methods for effecting contraception are disclosed. These formulations, kits and methods involve the delivery of progestins and estrogens in amounts personalized to the body weight and or the BMI of the women receiving the treatment.

### FIELD OF THE INVENTION

The invention pertains to the field of contraception and more specifically to formulations that deliver progestins and estrogens to women, the levels of which are personalized to the body weight or BMI of the women receiving the treatment.

### BACKGROUND OF THE INVENTION

The World Health Organization (WHO) has established weight categories based on Body Mass Index (BMI=weight in kilograms/height in meters²) as follows:

Underweight, BMI <18.5 kg/m²

Normal, BMI >18.5 kg/m² but <24.9 kg/m²

Overweight, BMI >25 kg/m² but <29.9 kg/m²

Obese, BMI >30 kg/m²

In the United States 64% of women are overweight or obese, with 36% being obese (J. Am. Med. Assoc. 307(5):491 (2012)). The unintended pregnancies worldwide are about 40% with 43% in Europe and 51% in North America (Studies in Family Planning 45(3):301 (2014).

### SUMMARY OF THE INVENTION

A first broad aspect of the invention features formulations comprising levonorgestrel that deliver an amount of levonorgestrel that is based on the weight of the women treated and that is within a range of amounts, with the maximum amounts being no higher than those amounts obtained using the equation

Maximum amount of LNG=−0.0084(woman's weight)²+5.1893(woman's weight)−375.79

and with the minimum amounts being no less than those amounts obtained using the equation

Minimum amount of LNG=−0.0041(woman's weight)²+2.5504(woman's weight)−184.36

wherein amounts are expressed in micrograms per day (μg/d) and the woman's weight is expressed in pounds. In certain embodiments, the formulation also comprises a SHBG binding ligand, e.g., an estrogen, e.g., ethinyl estradiol, and the amount of LNG is adjusted as described in more detail herein.

This first aspect of the invention also features a method of effecting contraception in a woman by internally administering to the woman levonorgestrel at a daily dose that is selected on the basis of her body weight wherein said daily dose of the progestin is within the range of Dₘᵢₙ and Dₘₐₓ wherein

Dₘᵢₙ=the lower of 90 or [(0.0041*X²)+(2.5504*X)−184.4];

Dₘₐₓ=[(0.0084*X²)+(5.1893*X)−375.8];

Dₘᵢₙ and Dₘₐₓ are minimum and maximum values in μg/d (+/−10%) of levonorgestrel or are contraceptively equivalent amounts of another progestin;

the progestin is coadministered with about 30 μg/d ethinyl estradiol or Dₘᵢₙ and Dₘₐₓ are adjusted if (A) (i) a different amount of ethinyl estradiol is coadministered, (ii) a different estrogen is coadministered or (iii) a non-estrogen SHBG binding ligand is coadministered and (B) the progestin is one that binds to SHBG;

X is the woman's body weight in pounds

or wherein the method delivers an amount of ethinyl estradiol that is greater or lesser than about 30 μg/d (including no ethinyl estradiol) and the amount of LNG is optionally adjusted to take into account the ethinyl estradiol, as described in the specification below;

or wherein a SHBG binding ligand other than an estrogen, or an estrogen other than ethinyl estradiol, is delivered in an amount that is equivalent to about 30 μ/d ethinyl estradiol or to such greater or lesser amount (including no ethinyl estradiol).

This first aspect of the invention also features a product or product line comprising a set of contraceptive products for women, wherein each set comprises one or more pharmaceutical dosage units for delivering a predetermined contraceptive amount of a progestin per day during a treatment period of at least 21 days; the predetermined contraceptive amount is based on each woman's weight category; each weight category is a range of between 5 pounds and 50 pounds; and the predetermined contraceptive amount of the progestin for each weight category is within the range of Dₘᵢₙ and Dₘₐₓ;

wherein

Dₘᵢₙ=the lower of 90 or [(0.0041*X²)+(2.5504*X)−184.4];

Dₘₐₓ=[(0.0084*X²)+(5.1893*X)−375.8];

Dₘᵢₙ and Dₘₐₓ are μg/d (+/−10%) of levonorgestrel or are contraceptively equivalent amounts of another progestin;

the progestin is coadministered with 30 μg/d ethinyl estradiol or Dₘᵢₙ and Dₘₐₓ are adjusted if (A) (i) a different amount of ethinyl estradiol is coadministered, (ii) a different estrogen is coadministered or (iii) a non-estrogen SHBG binding ligand is coadministered and (B) the progestin is one that binds to SHBG;

X is the woman's body weight in pounds

or wherein the method delivers an amount of ethinyl estradiol that is greater or lesser than about 30 μg/d (including no ethinyl estradiol) and the amount of LNG is optionally adjusted as described in the specification.

or wherein a SHBG binding ligand other than an estrogen, or an estrogen other than ethinyl estradiol, is delivered in an amount that is equivalent to about 30 μ/d ethinyl estradiol or to such greater or lesser amount (including no ethinyl estradiol).

This first broad aspect of the invention also features a method of manufacturing a line of contraceptive pharmaceutical products for effecting contraception in a population of women of varying weight and/or BMI, the method comprising: (A) analyzing the results of a clinical study of a progestin or progestin-estrogen contraceptive product in women of varying BMIs and/or body weights, the analysis including the steps of: (i) preparing a graph of BMI or body weight versus number of pregnancies that occurred at each BMI or body weight; (ii) selecting a minimum and a maximum acceptable pregnancy rate for all women and calculating Kd for the selected minimum and maximum acceptable pregnancy rates; (iii) stratifying the women into subpopulations based on BMI or body weight range; and (iv) using the calculated Kd values for the minimum and maximum acceptable pregnancy rates, calculating the progestin dose required to achieve pregnancy rates within the selected minimum and maximum acceptable rates for all BMI or body weight subpopulations of women, using data from (i); and (B) manufacturing one set of contraceptive products of the same delivery type (e.g., oral, transdermal, implant, or IUD or other depot) for each weight/BMI category that delivers the required dose for each weight/BMI subpopulation of women.

In any of the above-described methods or product lines, a required dose can be adjusted to increase statistical confidence based on standard deviation and, optionally, wherein the dose is selected from within the range of the originally calculated required dose and the higher adjusted dose. In certain embodiments, the required dose is adjusted by setting a minimum amount of progestin, e.g., an amount that is equivalent to 90 to 120 μg/d LNG. In certain embodiments, the required dose is adjusted to take into account the amount of an estrogen or other SHBG binding ligand.

In any of the above-described methods or product lines, a required dose is calculated for each weight or BMI category for two acceptable pregnancy rates, e.g., about 1.5% and about 3%, and the amount of progestin in each set of contraceptive products is within the range of required doses calculated for each weight/BMI category.

In any of the above-described methods or product lines, the amount per day of the SHBG binding ligand, e.g., an estrogen, e.g., ethinyl estradiol, can be varied during a treatment cycle or the amount of the progestin, e.g., levonorgestrel, can be varied during a treatment cycle, or both.

A second broad aspect of the invention features formulations, kits comprising such formulations, and methods utilizing such formulations for personalized contraception in women, whereby the formulations, kits, or methods deliver a contraceptively effective amount of a progestin, which amount is based on the body weight or BMI of the woman, and, optionally, an SHBG binding ligand such as, for example, an estrogen such as, for example, ethinyl estradiol.

This aspect of the invention also features formulations, kits comprising such formulations, and methods utilizing such formulations for personalized contraception in women, whereby the formulations, kits, or methods deliver ethinyl estradiol at about 30 μg per day (or an equivalent amount of another estrogen or other SHBG binding ligand) and varying amounts of a progestin based on the body weight of the woman equivalent to the amount of levonorgestrel (LNG) shown below,
1. for women up to 120 pounds, an LNG equivalent amount of 90 μg per day or higher, e.g., 90 to 150 μg per day;
2. for women weighing between 120 and 150 pounds, an LNG equivalent amount of 100 μg per day or higher, e.g., 100 to 240 μg per day;
3. for women weighing between 150 and 180 pounds, an LNG equivalent amount of 140 μg per day or higher, e.g., 140 to 350 μg/d;
4. for women weighing between 180 and 210 pounds, an LNG equivalent amount of 150 μg per day or higher, e.g., 170 to 400 μg/d;
5. for women weighing between 210 and 240 pounds, an LNG equivalent amount of 150 μg per day or higher, e.g., 170 to 400 μg/d;
6. for women weighing between 240 and 270 pound, an LNG equivalent amount of 200 μg per day or higher, e.g., 200 to 500 μg/d;
7. for women of 270 pounds or more, an LNG equivalent amount of 200 μg per day or higher, e.g., 200 to 500 μg/d

or wherein the formulations, kits, or methods deliver an amount of ethinyl estradiol that is greater or lesser than about 30 μg/d (including no ethinyl estradiol) and the amount of LNG (or LNG equivalent) is optionally adjusted as described in the specification, or wherein a SHBG binding ligand other than an estrogen, or an estrogen other than ethinyl estradiol, is delivered in an amount that is equivalent to about 30 μ/d ethinyl estradiol or such greater or lesser amount (including no ethinyl estradiol).

In certain embodiments of the aspect recited above, the levonorgestrel equivalent amount provides a 95.5% confidence level that there will not be more than 3 pregnancies per 100 women years (i.e., the pregnancy rate among users will not exceed 3% in any given 12 month period), said levonorgestrel equivalent amounts being, e.g.,
1. For women up to 120 pounds, LNG equivalent amounts of between 90 and 215 μg per day
2. For women weighing between 120 and 150 pounds, LNG equivalent amounts of 104 and 302 μg per day
3. for women weighing between 150 and 180 pounds, LNG equivalent amounts of between 154 and 375 μg per day
4. for women weighing between 180 and 210 pounds, LNG equivalent amounts of between 179 to 433 μg per day
5. for women weighing between 210 and 240 pounds, LNG equivalent amounts of between 156 to 476 μg per day
6. for women weighing between 240 and 270 pounds, LNG equivalent amounts of between 226 and 503 μg per day
7. for women of 270 pounds or more, LNG equivalent amounts of between 208 to 516 μg per day

or wherein the formulations, kits, or methods deliver an amount of ethinyl estradiol that is greater or lesser than about 30 μg/d (including no ethinyl estradiol) and the amount of LNG (or LNG equivalent) is optionally adjusted as described in the specification, or wherein a SHBG binding ligand other than an estrogen, or an estrogen other than ethinyl estradiol, is delivered in an amount that is equivalent to about 30 μ/d ethinyl estradiol or such greater or lesser amount (including no ethinyl estradiol).

This second aspect of the invention also features formulations, kits comprising such formulations, and methods utilizing such formulations for personalized contraception in women, whereby the formulations, kits or methods deliver ethinyl estradiol at about 30 μg per day and varying amounts of a progestin based on the body weight of the woman equivalent to the amount of levonorgestrel (LNG) shown below,
1. for women up to 120 pounds, an LNG equivalent amount between 90 and 120 μg per day
2. for women weighing between 120 and 150 pounds, an LNG equivalent amount between 105 and 215 μg per day
3. for women weighing between 150 and 180 pounds, an LNG equivalent amount between 150 and 365 μg per day
4. for women weighing between 180 and 210 pounds, an LNG equivalent amount between 150 and 365 μg per day
5. for women weighing between 210 and 240 pounds, an LNG equivalent amount between 150 and 365 μg per day
6. for women weighing between 240 and 270 pound, an LNG equivalent amount between 200 and 460 μg per day, and
7. for women of 270 pounds or more, an LNG equivalent amount between 200 and 460 μg per day

or wherein the formulations, kits, or methods deliver an amount of ethinyl estradiol that is greater or lesser than about 30 μg/d (including no ethinyl estradiol) and the amount of LNG (or LNG equivalent) is optionally adjusted as described in the specification, or wherein a SHBG binding ligand other than an estrogen, or an estrogen other than ethinyl estradiol, is delivered in an amount that is equivalent to about 30 μ/d ethinyl estradiol or such greater or lesser amount (including no ethinyl estradiol).

In certain embodiments of the formulations, kits, or methods of this second broad aspect of the invention, the progestin is levonorgestrel and ethinyl estradiol is delivered at a dose of 30 μg/d. In other embodiments, the progestin is levonorgestrel and ethinyl estradiol is delivered at a dose of <30 μg/d or >30 μg/d and the dose of levonorgestrel is adjusted to take into account the amount of ethinyl estradiol or wherein a different SHBG binding ligand is delivered at a dose that binds SHBG to the same or substantially the same extent as 30 μg/d ethinyl estradiol. In other embodiments, the progestin is levonorgestrel and no estrogen or other SHBG binding ligand is delivered and the dose of levonorgestrel is adjusted to take into account the absence of ethinyl estradiol or other SHBG binding ligand. In alternatives to these embodiments, a progestin other than levonorgestrel is delivered in levonorgestrel-equivalent doses, the doses not being adjusted to take into account the presence or absence of an estrogen or other SHBG binding ligand if the progestin is one that does not bind SHBG or that only poorly binds SHBG.

In certain embodiments of the formulations, kits, or methods of this second broad aspect of the invention, the amount per day of the SHBG binding ligand, e.g., an estrogen, e.g., ethinyl estradiol, is varied during a treatment cycle or the amount of the progestin, e.g., levonorgestrel, is varied during a treatment cycle, or both.

In any of the embodiments in the second broad aspect of the invention, the amount per of the progestin can be based on the woman's BMI instead of, or in addition to, the woman's weight.

A third broad aspect of the invention features a method of effecting contraception in a woman comprising: (a) determining the weight of the woman; (b) internally administering to the woman a progestin and an estrogen in accordance with the doses recited in the specification, including any of the dosing schedules recited in Example 2.

An embodiment of this aspect of the invention features method of effecting contraception in a woman comprising: (a) determining the weight of the woman; and (b) internally administering to the woman a progestin and an estrogen in accordance with the following schedule:

a progestin equivalent to 90 to 120 μg/d of levonorgestrel, e.g., 120 μg/d, and an estrogen equivalent to 30 (e.g., 0 to 40) μg/d of ethinyl estradiol if the woman weighs less than 130 pounds;

a progestin equivalent to 150 to 329 μg/d of levonorgestrel, e.g., 200 μg/d, and an estrogen equivalent to 30 (e.g., 0 to 40) μg/d of ethinyl estradiol if the woman weighs more than 130 pounds but less than 200 pounds; or

a progestin equivalent to 150-460 μg/d of levonorgestrel, e.g., 250 μg/d, and an estrogen equivalent to 30 (e.g., 0 to 40) μg/d of ethinyl estradiol if the woman weighs over 200 pounds.

This third aspect of the invention also features a method of effecting contraception in a woman comprising: (a) providing a contraceptive dosage form comprising a progestin; (b) calculating based on clinical studies a dose of the progestin that is predicted to result in a pregnancy rate of 3% or less for each of a plurality of weight categories or BMI categories; (c) determining the weight (or BMI) of the woman; and (d) administering to the woman the dosage form comprising the dose of the progestin that is predicted to result in a pregnancy rate of 3% or less for women in the woman's weight category and/or BMI category.

One embodiment of the third broad aspect of the invention features a method of effecting contraception in a woman having a body weight of 200 pounds or more, comprising administering to the woman: (i) 340 mg/d LNG or an equivalent amount of a different progestin, and 30 μg ethinyl estradiol or an equivalent amount of another estrogen; or (ii) 260 mg/d LNG or an equivalent amount of a different progestin, and 30 μg ethinyl estradiol or an equivalent amount of another estrogen; or (iii) 200 mg/d LNG or an equivalent amount of a different progestin, and 30 μg ethinyl estradiol (or an equivalent amount of another estrogen). In this embodiment, dose (i) is initially administered to the woman and if side effects develop then dose (ii) is administered instead and if side effects develop then dose (iii) is administered instead.

Another embodiment of the third broad aspect of the invention features a method of effecting contraception in a woman having a body weight of 200 pounds or more, comprising administering to the woman: (i) 420 mg/d LNG or an equivalent amount of a different progestin, and 20 μg ethinyl estradiol or an equivalent amount of another estrogen; or (ii) 330 mg/d LNG or an equivalent amount of a different progestin, and 20 μg ethinyl estradiol or an equivalent amount of another estrogen; or (iii) 220 mg/d LNG or an equivalent amount of a different progestin, and 20 μg ethinyl estradiol or an equivalent amount of another estrogen. In this embodiment, dose (i) is initially administered to the woman and if side effects develop then dose (ii) is administered instead and if side effects develop then dose (iii) is administered instead.

In various embodiments of this third broad aspect of the invention, the method is designed to deliver an amount of ethinyl estradiol that is greater or lesser than about 30 μg/d (including no ethinyl estradiol) and the amount of LNG (or LNG equivalent) is optionally adjusted to take into account the different amount(s) of ethinyl estradiol delivered. Alternatively, a SHBG binding ligand other than an estrogen, or an estrogen other than ethinyl estradiol, is delivered in an amount that is equivalent to 30 μ/d ethinyl estradiol or to such greater or lesser amount (including no ethinyl estradiol).

In the embodiments described above, the amount per day of the SHBG binding ligand, e.g., an estrogen, e.g., ethinyl estradiol, can be varied during a treatment cycle or the amount of the progestin, e.g., levonorgestrel, can varied during a treatment cycle, or both.

A fourth broad aspect of the invention features a method for effecting contraception in a woman by administering to the woman a pharmaceutical composition formulated to deliver ethinyl estradiol at about 30 μg per day (or an equivalent amount of another estrogen) and to deliver an amount of a progestin based on the potency of the progestin and on the body weight or the BMI of the woman,

wherein the amount of the progestin is equivalent to the amount of levonorgestrel (LNG) recited below,
1. for women weighing <120 pounds, 90 to 120 μg LNG per day,
2. for women weighing ≥120<150 pounds, 100 to 210 μg LNG per day,
3. for women weighing ≥150 and <180 pounds, 150 to 315 μg LNG per day,
4. for women weighing ≥180 and <210 pounds, 150 to 364 μg LNG per day,
5. for women weighing ≥210 and <240 pounds, 150 to 364 μg LNG per day,
6. for women weighing ≥240 and <270 pound, 200 to 460 μg LNG per day, and
7. for women weighing ≥270 pounds, 200 to 460 μg LNG per day,
   optionally wherein the dose range endpoints per weight category recited above are +/−10% or +/−5%

or wherein the method delivers an amount of ethinyl estradiol that is greater or lesser than about 30 μg/d (including no ethinyl estradiol) and the amount of LNG (or LNG equivalent) is optionally adjusted as described in the specification,

or wherein a SHBG binding ligand other than an estrogen, or an estrogen other than ethinyl estradiol, is delivered in an amount that is equivalent to about 30 μ/d ethinyl estradiol or to such greater or lesser amount (including no ethinyl estradiol).

In one embodiment of this fourth broad aspect of the invention, an amount at or near the high end of each dose range is administered to a woman based on her weight and if the woman experiences adverse effects associated with exogenous progestins, the amount of the progestin is reduced. In this embodiment, the amount of the progestin is not reduced to an amount that is less than the low end of the range for the woman's weight category.

In embodiments of this fourth broad aspect of the invention, the amount per day of the SHBG binding ligand, e.g., an estrogen, e.g., ethinyl estradiol, is varied during a treatment cycle or the amount of the progestin, e.g., levonorgestrel, is varied during a treatment cycle, or both.

A fifth broad aspect of the invention features the formulations, kits, methods, or products of any of the preceding four broad aspects and embodiments therein, wherein the progestin is levonorgestrel (LNG) unless otherwise indicated. In this aspect,

In embodiments of this fifth aspect, the amount of EE is less than about 30 μg per day and for every one μg per day reduction of EE delivered below about 30 μg per day, the amount of LNG delivered is increased by 2%.

In other embodiments of this fifth aspect, the amount of EE is greater than about 30 μg per day and for every one μg per day increase in EE delivery above about 30 μg per day, the amount of LNG delivered is decreased by 2%.

Also encompassed by this fifth broad aspect of the invention are formulations, kits, methods, or products of any of the preceding four broad aspects and embodiments therein, wherein the progestin has a binding affinity to SHBG which is less than about 20% of the binding affinity of testosterone to SHBG. Unless otherwise indicated, the progestin is selected from norgestimate, norelgestromin, magestrol acetate, drospirenone, medroxyprogesterone, norethynodrel, norethrindrone or lynestrenol, or combinations thereof.

In any of the aspects or embodiments of the formulations, kits, or methods described herein, the method of delivering the hormone(s) can be by oral administration, or by transdermal, implant, or injectable administration.

Other features and advantages of the invention will be appreciated by reference to the drawings and the detailed description and examples that follow.

### BRIEF DESCRIPTION OF THE FIGURES

FIG. 1 shows the percentage of women enrolled in a clinical study (Example 1) that became pregnant in each body weight category.

FIG. 2 shows the percentage of women enrolled in a clinical study (Example 1) that became pregnant in each BMI category.

FIG. 3 shows the doses of levonorgestrel required to achieve acceptable pregnancy rates of between 1.5 and 3 pregnancies per 100 enrolled women based on body weight, when co-administered with 30 μg/day of ethinyl estradiol.

FIG. 3a represents a statistical analysis which estimates the doses of levonorgestrel required to achieve 1.5% pregnancy rate based on body weight when co-administered with 30 μg/day of ethinyl estradiol.

FIG. 3b represents a statistical analysis which estimates the doses of levonorgestrel required to achieve a 3% pregnancy rate based on body weight when co-administered with 30 μg/day of ethinyl estradiol.

FIG. 4 shows the doses of levonorgestrel required to achieve acceptable pregnancy rates of between 1.5 and 3 pregnancies per 100 enrolled women based on BMI, when co-administered with 30 μg/day of ethinyl estradiol.

FIG. 4a represents a statistical analysis which estimates the doses of levonorgestrel required to achieve 1.5% pregnancy rate based on BMI when co-administered with 30 μg/day of ethinyl estradiol.

FIG. 4b represents a statistical analysis which estimates the doses of levonorgestrel required to achieve 3% pregnancy rate based on BMI when co-administered with 30 μg/day of ethinyl estradiol.

FIG. 5 shows the doses of levonorgestrel required to achieve acceptable pregnancy rates of between 1.5 and 3 pregnancies per 100 enrolled women based on body weight, with 95% confidence (two standard deviations), when co-administered with 30 μg/day of ethinyl estradiol.

FIG. 5a shows the doses of levonorgestrel required to achieve acceptable pregnancy rates of between 1.5 and 3 pregnancies per 100 enrolled women based on body weight, with 68% confidence (one standard deviation), when co-administered with 30 μg/day of ethinyl estradiol.

FIG. 6 shows the doses of levonorgestrel required to achieve acceptable pregnancy rates of between 1.5 and 3 pregnancies per 100 enrolled women based on BMI, with 95% confidence (two standard deviations), when co-administered with 30 μg/day of ethinyl estradiol.

FIG. 6a shows the doses of levonorgestrel required to achieve acceptable pregnancy rates of between 1.5 and 3 pregnancies per 100 enrolled women based on BMI, with 68% confidence (one standard deviation), when co-administered with 30 μg/day of ethinyl estradiol.

### DESCRIPTION OF ILLUSTRATIVE EMBODIMENTS OF THE INVENTION

There is an important unmet need pertaining to the determination of the amount of progestin required to provide contraceptive effectiveness to all women based on their BMI or body weight. Without intending to be bound to a particular understanding of mechanism of action, this invention is premised in part on the understanding that contraceptive effectiveness is based on the principle that there is a physical reaction or binding of a drug to its receptor and that therefore drug concentration will play an important part in contraceptive effectiveness. Further, by knowing the effect of a progestin amount (drug concentration) on contraceptive effectiveness on different BMI or body weight women, we can determine the amounts needed to provide effectiveness for any woman of known BMI or body weight. Given that contraceptive hormones interact reversibly with their receptors and assuming the resultant effect is proportional to the receptors occupied, a relationship between the effect and the free concentration of drug can be written as (Goodman and Gilman's Pharmaceutical Basis of Therapeutics, 8^{th} edition, p. 44)

Effect=Maximal effect (D)/(Kd+D) (equation 1)

Where D is the concentration of the circulating free drug and Kd is the dissociation constant for the drug-drug receptor complex, i.e., the ratio of the rate constants of the forward and dissociation binding reactions. The steady state concentration D of the circulating free drug in the plasma is correlated to the dosage rate, by the equation (Goodman and Gilman's Pharmaceutical Basis of Therapeutics, 8^{th} edition, p. 21), Dosage Rate=CL×D (CL is the clearance and it is constant for any specific drug). Therefore D=Dosage rate/CL. Substituting this in equation 1 one obtains

Effect=Maximal effect×Dosage rate/CL/(Kd+Dosage rate/CL) (equation 2)

wherein Maximal effect is 100% (i.e., pregnancy rate=0) and Effect is the actual efficacy rate for a given weight category (e.g., if pregnancy rate=1.5%, then Effect=98.5%).

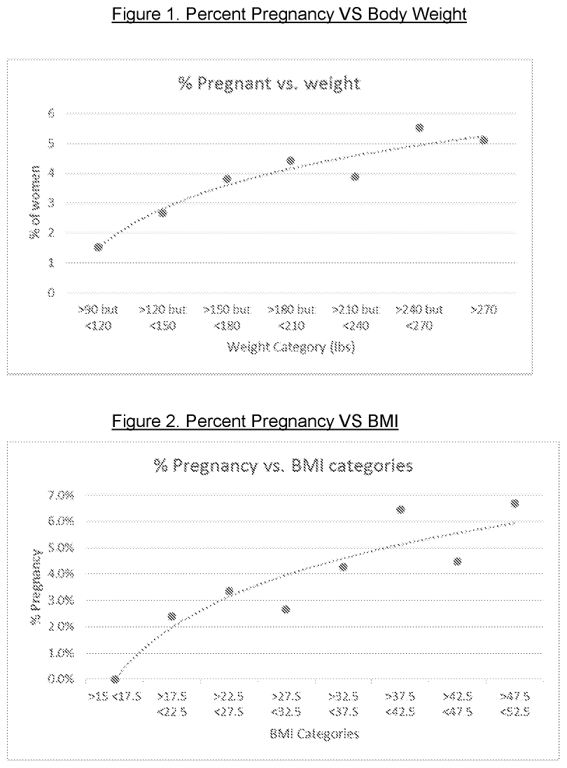

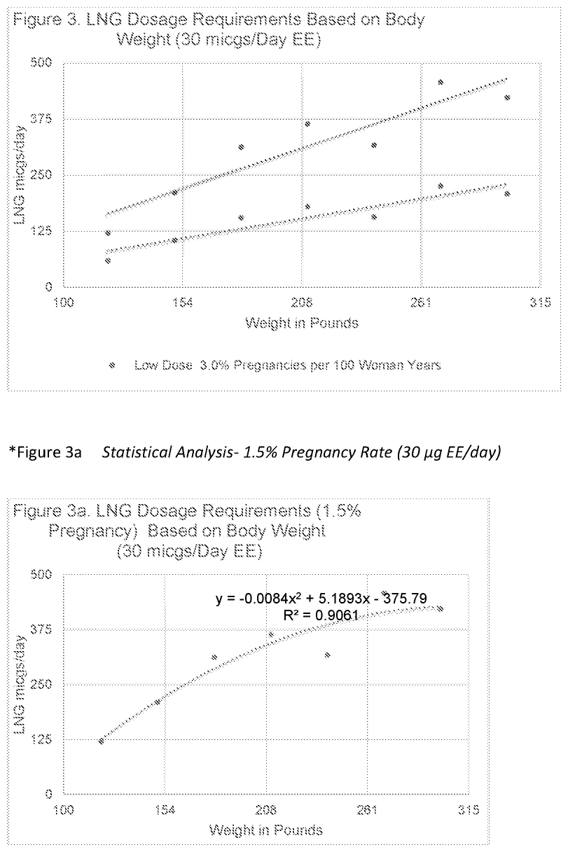

To obtain the required data that would allow us to determine the personalized dosage of progestin required for each woman, we performed a clinical trial which is shown below as Example 1. When administered 120 micrograms per day of levonorgestrel (LNG), the number of women in each body weight category and the percent of the women in each category that became pregnant over a period of one year is shown in Table 1 and graphically depicted in FIG. 1. Similar information based on BMI and percent that became pregnant is shown in Table 1a and FIG. 2.

TABLE 1
Weight Categories and % Pregnancies

| Weight Category | # of subjects | % of pregnancies |
|---|---|---|
| >90 but ≤ 120 | 199 | 1.51 |
| >120 but ≤ 150 | 563 | 2.66 |
| >150 but ≤ 180 | 473 | 3.81 |
| >180 but ≤ 210 | 295 | 4.41 |
| >210 but ≤ 240 | 150 | 3.87 |
| >240 but ≤ 270 | 91 | 5.49 |
| >270 | 59 | 5.08 |

TABLE 1a
BMI Categories and % Pregnancies

| BMI Category | # of Subjects | % of pregnancies |
|---|---|---|
| >15 < 17.5 | 18 | 0.00% |
| >17.5 < 22.5 | 378 | 2.38% |
| >22.5 < 27.5 | 597 | 3.35% |
| >27.5 < 32.5 | 378 | 2.65% |
| >32.5 < 37.5 | 235 | 4.26% |
| >37.5 < 42.5 | 140 | 6.43% |
| >42.5 < 47.5 | 67 | 4.48% |
| >47.5 < 52.5 | 15 | 6.67% |

Levonorgestrel Required Dosage Based on Women's Body Weight (30 Micrograms EE)

Looking at FIG. 1, “% pregnant VS body weight” and accepting the fact that the % pregnant of 1.5% for women of more than 90 pounds and less than 120 pounds is acceptable when the progestin dosage rate is 120 μg levonorgestrel (LNG) per day (and 30 microgram per day EE), then equation 2 becomes:

Kd=1.827/CL (equation 3)

Considering that 1.5% pregnancy is an acceptable reference level and knowing Kd, equation 2 can be used to solve for dosage rate for any level of effect for the levonorgestrel patch represented in the FIG. 1, “% pregnant VS body weight”. Note that when equation 3 is substituted into equation 2 the clearance CL cancels out and is not involved in the calculations.

Using the above formulas and isolating for D yields this equation:

D=(Kd*Effect)/(1−Effect) (equation 4)

The putative dosage levels of LNG were calculated based on the pregnancy rate for each weight category, using equations 2 and 3 and are shown in Table 2, column 2 under the title “LNG Level”. They represent the relative dosage compared to that of the women in the 90 to 120 pound category (i.e. those that had 1.5% pregnancy rate). The putative LNG required dosage rates (selecting the highest required dose on the curve for each weight category) are shown in column 3 in Table 2. By “required dose” is meant the dosage of LNG required to be delivered into the blood of women (based on their weight) in order to achieve the same or substantially the same efficacy as the LNG dosage of 120 micrograms per day in the reference weight category (in this case 90 to 120 pounds/1.5% pregnancy rate).

For example, in the 120 to 150 pounds category, the pregnancy rate was 2.66%. Therefore,

D=(1.827*0.973)/(0.027) (equation 5)

i.e.,

D=67 μg/day (equation 6)

and

Required Dose=120×120/67=215 (equation 7).

Results are plotted and regression analysis is performed.

Delivery of levonorgestrel dosage levels below those shown in Table 2 column 3 would result in percent pregnancy rates higher than 1.5%. Conversely, higher levels of levonorgestrel than those in Table 2, column 3 could result in lower than 1.5 percent pregnancy rates. Care should be taken to ensure that the higher levonorgestrel dosages are below those which would cause unacceptable side effects.

We consider the maximum acceptable percent pregnancy for 100 women over a period of one year to be 3%. The calculations mentioned above for the 1.5% pregnancy rate can be performed for the 3% pregnancy rate (Kd=3.705/CL) and are shown in columns 4 and 5 of Table 2.

TABLE 2
Levonorgestrel Dosage Requirements Based on Patient's Weight
Category (30 μg/day EE)

| | | | 3% | |
|---|---|---|---|---|
| Weight | 1.5% | | | Required |
| Category | LNG Level | Required Dose | LNG Level | Dose |
| (lbs) | (μg/day) | (μg/day) | (μg/day) | (μg/day) |
| 90 ≥ W <120 | 120.0 | 120 | 241.7 | 59 |
| 120 ≥ W <150 | 66.9 | 210 | 135.6 | 104 |
| 150 ≥ W <180 | 46.1 | 312 | 93.5 | 154 |
| 180 ≥ W <210 | 39.6 | 364 | 80.3 | 179 |
| 210 ≥ W <240 | 45.4 | 317 | 92.0 | 156 |
| 240 ≥ W <270 | 31.5 | 457 | 63.8 | 226 |
| 270 ≥ W | 34.1 | 422 | 69.2 | 208 |

Since 1.5 to 3 pregnancies per 100 enrolled women per year is an acceptable range of pregnancy rates for a clinical study of contraceptives, the amount of LNG to be delivered for each weight category to meet the above mentioned pregnancy rates of between 1.5% and 3% are shown in Table 3. Because the clinical study in which these results were obtained was a one year study, “100 enrolled women” (notwithstanding that not all subjects completed the study) can be considered a surrogate for “100 woman years.”

TABLE 3
Personalized Range of Levonorgestrel Daily Amount Required
for Effective Level of Contraception (EE 30 μg/day)

| Body Weight Category | | LNG Required Dose |
|---|---|---|
| 90 to <120 | pounds | 59 to 120 μg/d |
| ≥120 to <150 | pounds | 104 to 210 μg/d |
| ≥150 to <180 | pounds | 154 to 312 μg/d |
| ≥180 to <210 | pounds | 179 to 364 μg/d |
| ≥210 to <240 | pounds | 156 to 317 μg/d |
| ≥240 to <270 | pounds | 225 to 457 μg/d |
| 270 and more | pounds | 208 to 422 μg/d |

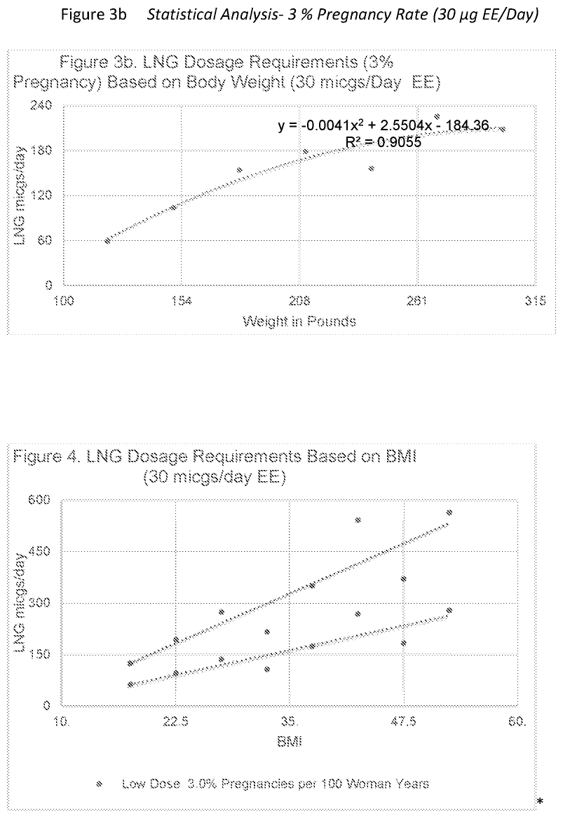

FIG. 3 graphically shows the amounts of LNG that will have to be delivered, i.e., the required dose, to women depending on their body weight. The area between the two curves represents the LNG delivery dosage rates that would allow for acceptable pregnancy rates of between 1.5 and 3 pregnancies per 100 enrolled women (indicated as “100 Woman Years” in the figure legend).

It is therefore an object of our invention to treat women in a personalized manner depending on their body weight, delivering the required amount of levonorgestrel as shown in Table 3 and FIG. 3.

In an illustrative personalized protocol, each woman will be initially treated at the highest level in her body weight category and the level reduced to the lowest level of her weight category if side effects are experienced due to the higher progestin level. In another illustrative embodiment, each woman is given a fixed dose within a range of doses determined to provide an acceptable pregnancy rate for women within her weight category.

Levonorgestrel Required Dosage Based on Women's BMI (30 Micrograms EE)

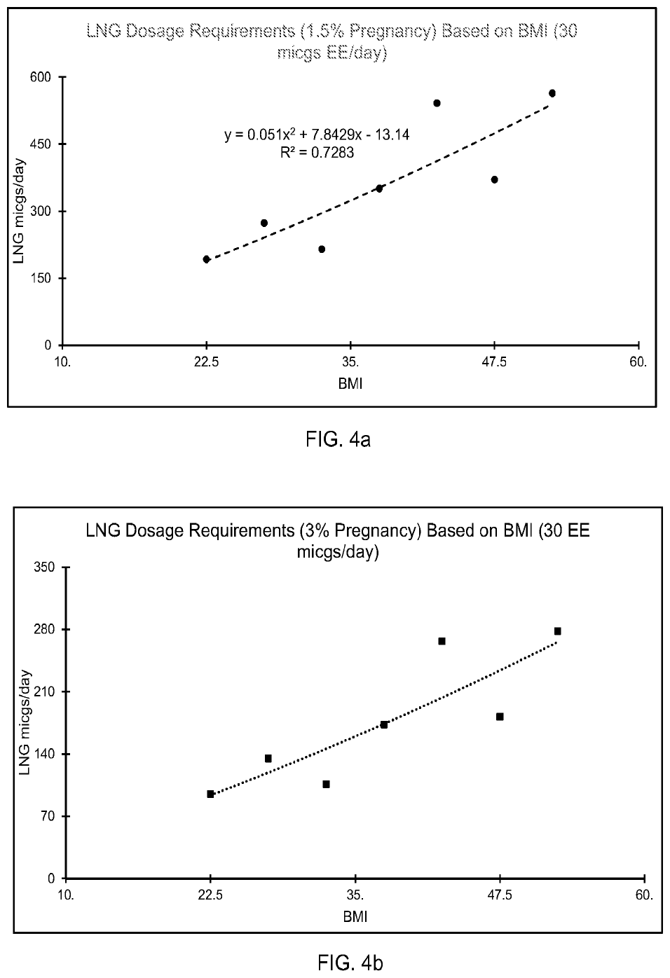

The analysis mentioned above was performed using the body weight of the women that were enrolled in the study. The same analysis can be performed using the BMI of the women. As can be seen in Table 1a and FIG. 2 under example 1, the relationship of the “% pregnant to BMI” is similar to that of “% pregnant to body weight”. The same analysis as performed for the required dosage based on women's body weight was performed using BMI and the data are shown in Tables 4 and 5 and plotted in FIG. 4. Although it may be easier for the medical practitioner to use the body weight of the subject in the treatment process, she can also use the BMI. It is therefore another object of the invention to treat women in a personalized manner depending on their BMI.

In FIG. 4, the LNG required doses are on the lines that best fit the data points.

TABLE 4
Levonorgestrel Dosage Requirements Based on Patient's BMI Category
(30 μg/day)

| | | | 3% | |
|---|---|---|---|---|
| | 1.5% | | | Required |
| | LNG Level | Required Dose | LNG Level | Dose |
| BMI Category | (μg/day) | (μg/day) | (μg/day) | (μg/day) |
| >15 ≤ 17.5 | — | — | — | — |
| >17.5 ≤ 22.5 | 74.7 | 192 | 152.0 | 95 |
| >22.5 ≤ 27.5 | 52.7 | 273 | 106.9 | 135 |
| >27.5 ≤ 32.5 | 67.1 | 215 | 136.1 | 106 |
| >32.5 ≤ 37.5 | 41.1 | 350 | 83.3 | 173 |
| >37.5 ≤ 42.5 | 26.6 | 541 | 53.8 | 267 |
| >42.5 ≤ 47.5 | 38.9 | 370 | 79 | 182 |
| >47.5 ≤ 52.5 | 25.6 | 563 | 51.8 | 278 |

TABLE 5
Personalized Range of Levonorgestrel Daily Dosage Required for
Effective Level of Contraception Based on Women's BMI (EE 30 ug/day)

| BMI Category | LNG Required Dose (μg/d) |
|---|---|
| >15 ≤ 17.5* | — |
| >17.5 ≤ 22.5 | 95-192 |
| >22.5 ≤ 27.5 | 135-273 |
| >27.5 ≤ 32.5 | 106-215 |
| >32.5 ≤ 37.5 | 173-350 |
| >37.5 ≤ 42.5 | 267-541 |
| >42.5 ≤ 47.5 | 182-370 |
| >47.5 ≤ 52.5 | 278-563 |

*No pregnancies in this BMI category.

Statistical Analysis of the Data Based on Women's Body Weight.

A review of the data points associated with Tables 2 and 3 reveal a relationship which could be linear or quadratic and since the data has substantial variability in the individual points a statistical analysis of the data was performed. The applicable linear and quadratic equations had correlation coefficients R² of 0.85 and 0.91 respectively and standard error of within 10 micrograms. Although the plots would be very similar we chose to present the data based on the quadratic equation since it had somewhat higher correlation coefficient. The applicable graphs and related statistical information for 1.5% and 3% pregnancy rates (30 μg EE), are as shown in FIGS. 3a and 3b for the data based on women's weight.

As indicated in FIG. 3a, the resulting implied equation is

Y=−0.0084X²+5.1893X−375.79

Where,

Y=the estimated required daily dose of LNG in μg/d

X=woman's weight in pounds

Moreover, the coefficient of determination, or R², is approximately 91% with a standard error of the estimate of 44.2 μg. Thus, the above equation could be modified as follows, to ensure that the correct amount of LNG was prescribed at the appropriate confidence levels (confidence limits),

Y=−0.0084X²+5.1893X−375.79+Z(44.2)

Where,

Z represents the applicable coefficient to obtain a desired level of confidence (e.g. a coefficient of one implies 68% confidence level, two would imply a 95.5% confidence level and a coefficient of three would imply a 99.7% confidence level; Z=0 implies that the values do not take into account the standard error and represent the exact values on the lines as in FIG. 3). The other variables are the same as indicated above. For example, the daily dose required for a 150 pound woman assuming a 95.5% confidence level would be determined as follows:

−0.0084(150)²+5.1893(150)−375.79+2(44.2)=302 μg

The 302 μg daily dose represents the high boundary value of levonorgestrel that could be prescribed to a 150 pound woman with a 95.5% confidence that this value will provide a pregnancy rate of 1.5% pregnancies per 100 woman years.

A similar analysis for the 3% pregnancy rate was performed and the data and resulting graph are shown in FIG. 3b.

As indicated for the 3% pregnancy rate in graph 3b, the resulting implied equation is

Y=−0.0041X²+2.5504X−184.36

Where,

Y=the estimated required dose of LNG in μg/d

X=woman's weight in pounds

Moreover, the coefficient of determination, or R², is approximately 91% with a standard error of the estimate of 21.8 μg. Thus, the above equation could be modified as follows, to ensure that the correct amount of LNG was prescribed at the appropriate confidence levels (confidence limits),

Y=−0.0041X²+2.5504X−184.36−Z(21.8)

Where,

Z represents the applicable coefficient to obtain a desired level of confidence (e.g. a coefficient of two would imply a 95.5% confidence level and a coefficient of three would imply a 99.7% confidence level). The other variables are the same as indicated above. For example, the dose required for a 150 pound woman assuming a 95% confidence level would be determined as follows:

−0.0041(150)²+2.5504(150)−184.36−2(21.8)=62.4 μg

The 62.4 μg daily dose represents the low boundary value of levonorgestrel that could be prescribed to a 150 pound woman with a 95.5% confidence that this value will provide a pregnancy rate of 3 pregnancies per 100 woman years.

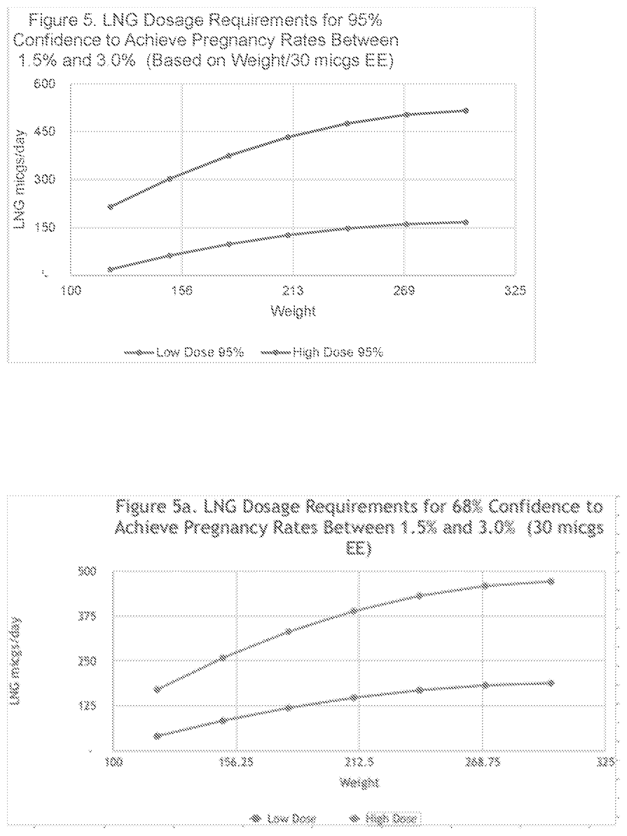

Table 6 and FIG. 5 summarize the maximum and the minimum dose of levonorgestrel levels required at the 95.5% level of confidence (two standard deviation units, i.e., two σ) for varying woman weight categories as determined by the modified equations described above.

TABLE 6
Daily LNG Required Dose Based on Weight for
95.5% Confidence Level (EE 30 mg)

| | 3% | 1.5% |
|---|---|---|
| | Pregnancy Rate | Pregnancy Rate |
| Weight | Dose (μg/d) | Dose (μg/d) |
| 90 to ≤120 | 19 | 215 |
| 120 to ≤150 | 62 | 302 |
| 150 to ≤180 | 98 | 375 |
| 180 to ≤210 | 126 | 433 |
| 210 to ≤240 | 147 | 476 |
| 240 to ≤270 | 161 | 503 |
| 270+ | 167 | 516 |

Table 6a and FIG. 5a summarize the maximum and the minimum dose of levonorgestrel levels required at the 68% level of confidence (one standard deviation unit, i.e., one σ) for varying woman weight categories as determined by the modified equations described above.

TABLE 6a
Daily LNG Required Dose Based on Weight for
68% Confidence Level (EE 30 mg)

| | 3% | 1.5% |
|---|---|---|
| | Pregnancy Rate | Pregnancy Rate |
| Weight | Dose (μg/d) | Dose (μg/d) |
| 90 to ≤120 | 41 | 171 |
| 120 to ≤150 | 84 | 258 |
| 150 to ≤180 | 120 | 331 |
| 180 to ≤210 | 148 | 389 |
| 210 to ≤240 | 169 | 432 |
| 240 to ≤270 | 183 | 459 |
| 270+ | 189 | 472 |

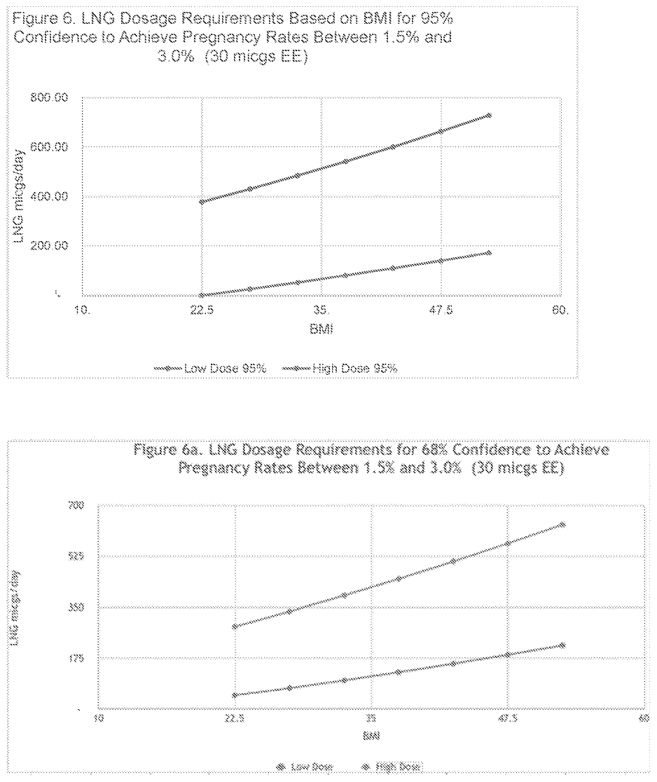

Table 7 and FIG. 6 summarize the maximum and the minimum dose of levonorgestrel levels required at the 95.5% level of confidence (two standard deviation units, i.e., two σ) for varying woman BMI categories.

TABLE 7
LNG Daily Required Dose Based on BMI, for 95.5% Confidence Level
(30 μg EE)

| | 1.5% Pregnancy | 3% Pregnancy |
|---|---|---|
| BMI | Rate (μg) | Rate (μg) |
| >17.5 < 22.5 | 378 | 0.1 |
| >22.5 < 27.5 | 430 | 26 |
| >27.5 < 32.5 | 484 | 52 |
| >32.5 < 37.5 | 541 | 80 |
| >37.5 < 42.5 | 601 | 110 |
| >42.5 < 47.5 | 663 | 140 |
| >47.5 < 52.5 | 728 | 172 |

Table 7a and FIG. 6a summarize the maximum and the minimum dose of levonorgestrel levels required at the 68% level of confidence (one standard deviation unit, i.e., one σ) for varying woman BMI categories.

TABLE 7a
LNG Daily Required Dose Based on BMI, for 68% Confidence Level
(30 μg EE)

| | 1.5% Pregnancy | 3% Pregnancy |
|---|---|---|
| BMI | Rate (μg) | Rate (μg) |
| >17.5 < 22.5 | 283 | 47 |
| >22.5 < 27.5 | 335 | 72 |
| >27.5 < 32.5 | 390 | 99 |
| >32.5 < 37.5 | 447 | 127 |
| >37.5 < 42.5 | 507 | 157 |
| >42.5 < 47.5 | 569 | 187 |
| >47.5 < 52.5 | 633 | 219 |

Levonorgestrel Required Dosage at Different Ethinyl Estradiol Levels

FIGS. 3, 3a and 3b summarize the required levels of levonorgestrel when the ethinyl estradiol (EE) co-administered with LNG, is about 30 μg per day. However, ethinyl estradiol binds to SHBG and in the process it releases some of the bound LNG. Therefore if more or less than about 30 μg of EE are delivered from the contraceptive formulation, the required levels shown in FIGS. 3, 3a and 3b will have to be modified accordingly. Patent application PCT/US2016/033024 (WO2016187269) states “As seen from the examples below, the inventors have experimentally determined that for every 10 μg per day of EE delivered, the amount of free LNG circulating in the plasma is increased by 300 picograms per ml without increasing the amount of levonorgestrel delivered”. This level corresponds to 20% of the approximate steady state level of 1500 picograms LNG per ml. Therefore the levels of required LNG can be calculated for any level of accompanied EE. Table 8 summarizes the LNG requirements for two formulations containing respectively 20 μg or 40 μg EE. Although the above discussion pertains to EE, other estrogenic compounds such as estradiol, mestranol, estrone, estriol, isoflavones or coumestans can be used at EE equivalent amounts, i.e., amounts that have similar effects on estrogen receptor, on SHBG, or on both. As used herein, “about” generally means +/−10% so, e.g., “about 30 μg” means 27 to 33 μg.

TABLE 8
Personalized Range of LNG Daily Amount Required for Effective Level of
Contraception Based on a Woman's Weight (EE 20 or 40 μg/day)

| | LNG Required Dose | LNG Required Dose |
|---|---|---|
| | (μg/d) | (μg/d) |
| Body Weight Category | (20 μg EE/day) | (40 μg EE/day) |
| 90 to ≤120 pounds, | 71 to 144 | 47 to 96 |
| >120 to ≤150 pounds, | 144 to 252 | 83 to 168 |
| >150 to ≤180 pounds, | 185 to 374 | 123 to 250 |
| >180 to ≤210 pounds, | 215 to 435 | 143 to 291 |
| >210 to ≤240 pounds, | 187 to 380 | 125 to 254 |
| >240 to ≤270 pounds, | 270 to 548 | 180 to 366 |
| >270 | 250 to 506 | 166 to 338 |

The data shown in Table 8 correspond to those in Table 3 but increased or decreased by 20% for co-delivered levels of EE of 20 micrograms or 40 micrograms respectively. Table 7 does not include any confidence levels, i.e. Z=0. LNG required dosage levels for 95% confidence for 20 and 40 micrograms EE can be calculated by increasing or decreasing the LNG dosage levels in Table 6, by 20% (data not shown). Therefore, it is another object of our invention to provide levonorgestrel equivalent dosage levels for any level of co-administered EE.

This invention therefore comprises embodiments in which no estrogen is present and in which only small amounts of an estrogen are present, e.g., <10 μg/d of ethinyl estradiol, as disclosed in WO2016187269. Other embodiments comprise one or more SHBG binding ligands other than or in addition to an estrogen or a progestin, i.e., a non-progestin binding ligand, also as disclosed in WO2016187269. As illustrated in WO2016187269, e.g., FIG. 1, the relationship between progestin plasma levels and amount of ethinyl estradiol delivered is linear, making it straightforward to calculate an appropriate adjustment in the dose of progestin for any dose of ethinyl estradiol. In illustrative embodiments of the invention, if the progestin is LNG and, if the amount of EE co-delivered is less than 30 μg per day, then for every one μg per day reduction of EE delivered below 30 μg per day, the amount of free LNG in the plasma is decreased by 30 picograms per ml and, if the amount of EE co-delivered is greater than 30 μg per day, then for every one μg per day increase in EE delivery above 30 μg per day, the amount of free LNG in the plasma is increased by 30 picograms per ml.

On the other hand, some progestins do not bind or bind only poorly to SHBG (e.g., less than 20% binding affinity to SHBG when compared to the affinity of testosterone to SHBG). See, the discussion below and also WO2016187269. Therefore, circulating amounts of such progestins are not affected by the presence of an estrogen (or other SHBG binding ligand) and the dose of such progestins need not be modified to take into account the presence or absence of an estrogen. So, e.g., a contraceptive product that delivers 150 μg/d norelgestromin to “low weight women” (e.g., <150 lbs or BMI <25), 225 μg/d norelgestromin to “medium weight women” (e.g., 150 to <250 lbs or BMI <30), and 335 μg/d norelgestromin to “high weight women” (e.g., ≥250 lbs or BMI ≥30) would comprise and deliver approximately the same amount of the progestin when delivered in combination with 20 μg/d ethinyl estradiol, 30 μg/d ethinyl estradiol, 40 μg/d ethinyl estradiol, or 0 μg/d ethinyl estradiol.

The discussion, above, is from the perspective of delivery of a fixed amount of EE and LNG during the entirety of a given treatment interval. However, the skilled person will understand that the amount per day of EE, or of another estrogen, or of another SHBG binding ligand can be varied during a treatment interval. So, for example, the amount of EE may be varied, e.g., day to day or week to week, during a treatment interval, e.g., 5 to 40 μg/d. In this case, the amount of LNG to be delivered is optionally adjusted based on the amount of EE as discussed above. So, e.g., if the change in EE delivered is small, or if an effect on free progestin levels is desired, then it is not necessary to adjust the amount of the progestin.

Similarly, the EE can be administered in combination with a different SHBG binding ligand, or an SHBG binding ligand other than EE can be substituted for EE for all or a portion of a treatment interval.

Similarly, the amount of LNG or other progestin can be varied during a given treatment interval, typically within the ranges for an applicable weight or BMI category, calculated as described herein.

To be clear, the amount of EE or LNG or both can be varied from day to day or week to week. EE can be varied from 5 to 40 μ/day and LNG can also be varied as long as the amount delivered is within the levels described in the specification section and the examples, for the specific body weight or BMI category. The amount of EE and LNG can also be varied in the drug free interval if EE and LNG are also delivered during that interval (U.S. Pat. Nos. 9,198,920, 9,198,919, 9,198,876, 9,192,614).

Statistical Analysis of the Data Based on Women's BMI.

A review of the data points associated with Tables 4 and 5 reveal again a relationship which could be linear or quadratic. Again, since the data has substantial variability in the individual points a statistical analysis of the data was performed. The applicable linear and quadratic equations had correlation coefficients R2 of 0.73 for both 1.5 and 3% pregnancy rates. The standard error for the 1.5% pregnancy rate (linear VS quadratic) as well as the 3% pregnancy rate (linear versus quadratic) was within 10 micrograms of each other. The correlation coefficients are substantially lower than those which are based on body weight, indicating that the LNG required dose is better related to body weight than BMI. We chose to draw FIGS. 4a and 4b based on the quadratic equations, so as to have an easier comparison to the corresponding body weight lines in FIGS. 3a and 3b.

As indicated above the implied equation is shown below, where the symbols Y and X are the same as presented above. The correlation coefficient R2 is 0.73 and the standard error 94.3 micrograms.

Y=0.051X²+7.8429X−13.14

As discussed previously the above equation can be modified to provide a 95.5% confidence level, i.e. to insure that the correct amount of LNG was prescribed for that confidence level. That is

Y=0.051X²+7.8429X−13.14+188.6

For example the LNG daily dose required for a woman with BMI of 27.5, assuming a 95.5% confidence level, will be determined as follows

0.051(27.5)²+7.8429(27.5)−13.14+188.6=430 micrograms

The 430 microgram of LNG daily dosage represents the high boundary value of levonorgestrel that could be prescribed to a 27.5 BMI woman with a 95.5% confidence that this value will provide a pregnancy rate of 1.5% pregnancies per 100 woman years.

A similar analysis for the 3% pregnancy rate was performed and the results are shown in FIG. 4b.

The resulting equation from FIG. 4b is

Y=0.0257X²+3.8143X−5.1964

Where, Y is the estimated required dose of LNG in micrograms and X is the woman's weight in pounds.

The correlation coefficient, R2 is 0.73 and the standard error 46.8. The above equation can be modified to provide a 95.5% confidence level as follows

Y=0.0257X²+3.8143X−98.8

For example the dose required for a 25 BMI woman, assuming a 95% confidence level would be

0.0257(25)²+3.8143(25)−98.8=26

Table 7 and FIG. 6 summarize the maximum and minimum dose of levonorgestrel levels required at the 95% confidence for varying woman BMI categories as determined from the modified equations described above.

As can be seen in FIGS. 3, 4, 5 and 6, the LNG daily dosage required to achieve pregnancy rates of between 1.5 and 3% per 100 women years, increases as the body weight or the BMI of the women increases. Therefore another practical approach is to treat all women with body weight of over 200 pounds with the same formulations. For example for women co-administered 30 μg EE per day, the level of LNG equivalent would be 350 μg per day (for the 1.5% pregnancy level), and 200 μg per day (for the 3% pregnancy level). An intermediate level of 260 μg per day can also be prepared.

Therefore it is another object of the invention to treat women of over 200 pounds by producing three dosage forms, such as pills containing respectively a) first pill 350 μg LNG, 30 μg EE, b) second pill 275 μg LNG, 30 μg EE, c) third pill 200 μg LNG, 30 μg EE. The doctor may initially prescribe to the overweight woman pill (a). If side effects develop due to the higher amount of progestin delivered, the doctor may optionally prescribe pill (b). If still side effects due to the progestin amount persist, the doctor may optionally prescribe pill (c). The same dosage forms could be administered for overweight women that are on 20 μg per day EE, except the LNG dosage levels would be respectively for pill a) 420 μg LNG, b) 330 μg LNG and c) 240 μg LNG (Z=0). For high BMI women similar formulations can be prepared, for example for women with BMI above 40 the three LNG dosage levels could be a) 400 μg LNG, b) 340 μg LNG and c) 275 μg LNG.

As mentioned above, progestins (as well as estrogens) bind to SHBG and have the ability to displace other hormones that are bound to it. Thus progestins have the ability to displace testosterone and dihydrotestosterone from SHBG thus allowing these free hormones to circulate into the blood. Testosterone and dihydrotestosterone are androgens which can cause androgenic side effects to women, such as increase in facial and body hair, acne or oily skin, menstrual irregularity, masculine physical qualities including male pattern baldness and other. Therefore progestin molecules with lower binding affinity to SHBG would be preferred with our invention. The relative binding affinity of steroids to SHBG (testosterone being 100) has been summarized by Westphal (Steroid-Protein Interactions II, Springer-Verlag, p. 256 (1986)) and others. Progestins with less than 20% binding comparative affinity to SHBG include medroxyprogesterone, lynestrerol, norethynodrel, norethindrone, I-norgestrel, norgestimate, drospirenone, norelgestromin and megestrol acetate. It is therefore another object of our invention to use in the contraceptive formulations of the invention, progestin molecules that have less than 20% binding affinity to SHBG when compared to the affinity of testosterone to SHBG.

A person skilled in the art can determine how much of another progestin, or combination of progestins, to substitute for levonorgestrel in the contraceptive formulations of the invention, based on known characteristics of levonorgestrel and the other selected progestins. Parameters useful to determine equivalent dosages of another progestin as compared with levonorgestrel include, but are not limited to, potency, bioavailability (via selected route of administration) and/or SHBG binding affinity.

Equivalent concentrations of estrogens and of progestins can be also determined using in vitro or in vivo assays. See, for example, Kuhl, H., Drugs 51(2):188-215 (1996); Philibert, D., et al., Gynecol. Endocrinol. 13:316-326 (1999); and Lundeen, S., et al., J. Steroid Biochem. Molec. Biol. 78:137-143 (2001), in which the relative potencies of various progestins are compared using both in vitro and in vivo assays. See also, for example, Dickey, R. P., “Contraceptive Therapy,” OBG Management Supp (October 2000), pp. 2-6.

One can also look to marketed contraceptive products to determine approximate equivalent amounts. For example, oral contraceptive products approved in the U.S. include the following combinations with 30 μg ethinyl estradiol:
0.15 mg desogestrel+0.03 mg ethinyl estradiol;
0.15 mg levonorgestrel+0.03 mg ethinyl estradiol;
3 mg drospirenone+0.03 mg ethinyl estradiol;
1.5 mg norethindrone acetate+0.03 mg ethinyl estradiol;

and the following combinations with 20 μg ethinyl estradiol:
3 mg drospirenone+0.02 mg ethinyl estradiol;
0.1 mg levonorgestrel+0.02 mg ethinyl estradiol;
1 mg norethindrone acetate+0.02 mg ethinyl estradiol.

From the above information, one can see that for a given dose of LNG, e.g., 120 μg/d, one might substitute 120 μg/d desogestrel, 1.0 mg/d norethindrone acetate, or 2.0 mg/d drospirenone. A person skilled in the art can similarly determine equivalent amounts of estrogens other than ethinyl estradiol.

The only transdermal product approved in the U.S. delivers 0.15 mg/d norelgestromin and 0.035 mg/d ethinyl estradiol from which it can be inferred that 120 μg/d LNG is approximately equivalent to 150 μg/d norelgestromin.

The doses described herein are based on the amounts of progestin and estrogen delivered per day during successive treatment cycles of three weeks on (“treatment interval”) and one week off (“rest interval”), although different treatment regimens can be employed.

Although oral formulations are preferred with the invention, other drug delivery approaches can be used, such as transdermal (passive, iontophoretic, microneedle), transmucosal (including but not limited to intravaginal), or by injection.

For oral delivery, the amount of hormones per dosage unit (i.e., per pill) is approximately the required dose as discussed herein. So, e.g., if the required dose of LNG is 90 μg/d with 30 μg/d EE, then each pill would typically comprise 90 μg/d LNG and 30 μg/d EE. For hormones with high metabolic rate in the liver appropriate adjustments will need to be made. A method is also envisioned where the subject is administered the highest level of LNG for her weight category. If side effects develop due to higher amount of progestin, the level can be reduced to an acceptable level where side effects are minimized or eliminated. However, the amount of progestin (e.g. LNG) administered should not drop below the lowest level for her weight category, otherwise the risk of getting pregnant will increase above the 3 percent pregnancy rate per 100 enrolled women.

It will be apparent that the invention as described above is illustrative and not limiting. So, e.g., one could employ different weight categories, e.g., <110 pounds, ≥110 to <130 pounds, ≥130 to <150 pounds, ≥150 to <170 pounds, ≥170 to 190 pounds, ≥190 to 210 pounds, ≥210 to 230 pounds, ≥230 to 250 pounds, and ≥250 pounds or <125 pounds, ≥125 to <150 pounds, ≥150 to <175 pounds, ≥175 to <200 pounds, ≥200 to 225 pounds, ≥225 to 250 pounds, ≥250 to 275 pounds, ≥275 to 300 pounds, and ≥300 pounds. It is essential to select a weight category for which the pregnancy rate does not exceed a maximum acceptable rate, which as illustrated above can be 1.5% or 3% or it can be another rate that a particular manufacturer or healthcare provider deems acceptable, e.g., 2%, 2.5%, 3%, or even 3.5%. It is unlikely that a pregnancy rate of greater than about 3% would be acceptable for the general population but it could be acceptable for a subpopulation of women, e.g., women prone to adverse effects associated with exogenous progestins.

As discussed above, it is also possible to use BMI instead of weight and to select and administer doses based on BMI category (e.g., see FIGS. 4 and 6 and tables 5 and 8), e.g., <18.5 kg/m², ≥18.5 kg/m² to <25 kg/m², ≥25 kg/m² to <30 kg/m², and ≥30 kg/m² or <18.5 kg/m², ≥18.5 kg/m² to <22 kg/m², ≥22 kg/m² to <25 kg/m², ≥25 kg/m² to <28 kg/m² and ≥30 kg/m².

With respect to dosing regimen, one can refer to numerous publications including, e.g., U.S. Pat. Nos. 9,198,876, 9,192,614, 9,198,919, and 9,198,920, including, e.g., use of low dose progestin, low dose estrogen, or low doses progestin and estrogen during rest intervals.

As stated above, pharmaceutical compositions of the present invention can be formulated for administration via a variety of routes known to the person of skill in the art, including oral, transmucosal (e.g., sublingual, thin film) and transdermal. The compositions can also be formulated within long-acting reversible contraceptive (LARC) devices, such as intrauterine devices (IUDs) and implants. Oral and sublingual dosage may be particularly suitable for delivery of SHBG ligands having a lesser binding affinity for SHBG than, for instance EE or 17 β-estradiol, since larger amounts of such ligands may be needed that cannot be effectively delivered by other routes.

Pharmaceutical formulations or preparations containing the compositions of the invention and a suitable carrier can be solid dosage forms which includes tablets, capsules, cachets, pellets, pills, powders or granules; topical dosage forms which include solutions, powders, fluid emulsions, fluid suspensions, semi-solids, ointments, pastes, creams, gels or jellies, foams and controlled release depot entities; transdermals, vaginal rings, buccal formulations; and implants.

It is known in the art that active ingredients are formulated into compositions with pharmaceutically acceptable diluents, fillers, disintegrants, binders, lubricants, surfactants, hydrophobic vehicles, water soluble vehicles, emulsifiers, buffers, humectants, moisturizers, solubilizers, antioxidants preservatives and the like. Numerous pharmacologic references are available for guidance, e.g., “Modern Pharmaceutics”, Banker & Rhodes, Marcel Dekker, Inc. (1979); “Goodman & Gilman's The Pharmaceutical Basis of Therapeutics”, 8th Edition, MacMillan Publishing Co., New York (1980), or Remington's Pharmaceutical Sciences, Osol, A., ed., Mack Publishing Company, Easton, Pa. (1980).

Transdermal compositions are formulated in accordance with well known methods, depending on the selected hormones to be delivered. In an exemplary embodiment, LNG is delivered from a transdermal delivery system comprising an adhesive polymer matrix and one or more skin permeation enhancers and other excipients as described in the Examples (see also U.S. Pat. Nos. 7,045,145 and 7,384,650). Delivery of other progestins can also be accomplished, with or without the use of skin permeation enhancers (see, e.g., WO 2013/112806 A2).

The compositions of the invention are preferably produced in the form of a kit or package, with the daily (e.g., for oral) or weekly (e.g., for transdermal) dosages arranged for proper sequential administration. Thus, other illustrative embodiments of the invention provide a pharmaceutical package that contains the contraceptive compositions in multiple dosage units in a synchronized, fixed sequence, wherein the sequence or arrangement of the dosage units corresponds to the stages of daily or weekly administration. In certain embodiments, such kits or packages contain placebos or low dose forms for use during a withdrawal interval between contraceptive treatments. These are referred to herein as “rest intervals” between “treatment intervals,” collectively comprising a “treatment cycle.” The placebos or low dose forms can take any form, including a different size or color of dosage form (e.g., pill or patch) that contains no contraceptively effective amounts of components. Alternatively, the package can contain “blanks,” such as, for instance, seven out of 28 blisters in a blister pack of oral dosage forms, or one out of four compartments in a transdermal package, being empty.

LARC devices, such as IUDs and implants, are typically formulated to contain only progestin. These devices can be supplemented with a non-progestin SHBG ligand to increase the amount of circulating progestin delivered from the devices and increase their efficacy.

The following non-limiting examples are provided to describe the invention in greater detail.

### EXAMPLES

### Example 1

In a clinical trial, 2032 healthy women, aged 18 and over, were enrolled in 102 investigative sites in the US. It was an open label, 13 cycle trial (one year) to study the effectiveness of a transdermal patch delivering 120 μg per day of levonorgestrel (LNG) and 30 μg per day of ethinyl estradiol (EE). The treatment regimen for each cycle was three consecutive 7-day patches (21 days) followed by one patch-free week.

The women enrolled in the different weight categories (in pounds) are shown in the second column of Table 1.

The percentage of women that became pregnant over the 13 cycle period is shown in column 3 of Table 1. A graph of the percentage of women that became pregnant in each weight category is also graphically illustrated in FIG. 1.

The same analysis was performed using the BMI of the women enrolled in the trial. The applicable data are plotted in FIG. 2 and the % pregnancies on the curve for each BMI category are listed in Table 1a.

The related graph of % pregnant versus BMI is shown in FIG. 2.

### Example 2

Methods of the invention are carried out by administering a progestin and an estrogen wherein the progestin is LNG administered in the following amounts and wherein the estrogen is ethinyl estradiol administered at 30 μg/d. Alternatively the progestin is a different progestin administered in an LNG-equivalent amount, the estrogen is a different estrogen delivered in an ethinyl estradiol-equivalent amount, and/or the ethinyl estradiol or other estrogen are administered in higher or lower amounts and the amount of the progestin is adjusted as described above.

Constructive Product 1:

| Body Weight Category | LNG Dose (μg/day) |
|---|---|
| up to 120 pounds, | 100 to 120 |
| 120 to <150 pounds, | 105 to 215 |
| 150 to <180 pounds, | 150 to 315 |
| 180 to <210 pounds, | 180 to 365 |
| 210 to <240 pounds, | 180 to 320 |
| 240 to <270 pounds, | 225 to 460 |
| 270 and more pounds, | 225 to 460. |

Constructive Product 2:

| Body Weight Category | LNG Dose (μg/day) |
|---|---|
| 90 to <120 pounds, | 100 to 120 |
| 120 to <150 pounds, | 100 to 260 |
| 150 to <180 pounds, | 120 to 335 |
| 180 to <210 pounds, | 145 to 390 |
| 210 to <240 pounds, | 165 to 435 |
| 240 to <270 pounds, | 180 to 460 |
| 270 and more pounds, | 185 to 475 |

Constructive Product 3:

| Body Weight Category | LNG Dose (μg/day) |
|---|---|
| 90 to <120 pounds, | 100 to 215 |
| 120 to <150 pounds, | 100 to 313 |
| 150 to <180 pounds, | 100 to 375 |
| 180 to <210 pounds, | 126 to 433 |
| 210 to <240 pounds, | 147 to 476 |
| 240 to <270 pounds, | 161 to 503 |
| 270 and more pounds, | 167 to 516 |

Constructive Product 4:

| Body Weight Category | LNG Dose (μg/day) |
|---|---|
| 90 to <120 pounds, | 100 to 125 |
| 120 to <150 pounds, | 100 to 220 |
| 150 to <180 pounds, | 150 to 315 |
| 180 to <210 pounds, | 175 to 375 |
| 210 to <240 pounds, | 175 to 425 |
| 240 to <270 pounds, | 175 to 475 |
| 270 and more pounds, | 200 to 475 |

Constructive Product 5:

| Body Weight Category | | LNG Dose (μg/day) |
|---|---|---|
| 150 | pounds, | 120 |
| 150 to 210 | pounds, | 240 |
| >210 | pounds, | 360 |

Constructive Product 6:

| Body Weight Category | | LNG Dose (μg/day) |
|---|---|---|
| 180 | pounds, | 120 |
| 180 to 240 | pounds, | 240 |
| >240 | pounds, | 360 |

Constructive Product 7:

| Body Weight Category | | LNG Dose (μg/day) |
|---|---|---|
| 120 | pounds, | 120 |
| 120 to <180 | pounds, | 200 |
| 180 to 270 | pounds, | 275 |
| 270 and more | pounds, | 350 |

Constructive Product 8:

| Body Weight Category | | LNG Dose (μg/day) |
|---|---|---|
| 150 | pounds, | 120 |
| 150 to 250 | pounds, | 180 |
| >250 | pounds, | 230 |

Constructive Product 9:

| BMI Category | LNG Required Dose (μg/d) |
|---|---|
| ≤22.5 | 100-192 |
| >22.5 ≤ 27.5 | 135-273 |
| >27.5 ≤ 32.5 | 106-215 |
| >32.5 ≤ 37.5 | 173-350 |
| >37.5 ≤ 42.5 | 267-541 |
| >42.5 ≤ 47.5 | 182-370 |
| >47.5 ≤ 52.5 | 278-563 |

Constructive Product 10:

| BMI Category | LNG Dose (μg/day) |
|---|---|
| ≤22.5 | 95-378 |
| >22.5 ≤ 27.5 | 135-430 |
| >27.5 ≤ 32.5 | 106 to 484 |
| >32.5 ≤ 37.5 | 173-541 |
| >37.5 ≤ 42.5 | 267-601 |
| >42.5 ≤ 47.5 | 182-663 |
| >47.5 ≤ 52.5 | 278-728 |

Constructive Product 11:

| BMI Category | LNG Dose (μg/day) |
|---|---|
| ≤22.5 | 95-283 |
| >22.5 ≤ 27.5 | 135-335 |
| >27.5 ≤ 32.5 | 106-390 |
| >32.5 ≤ 37.5 | 173-447 |
| >37.5 ≤ 42.5 | 267-507 |
| >42.5 ≤ 47.5 | 182-569 |
| >47.5 ≤ 52.5 | 278-633 |

Constructive Product 12:

| BMI Category | LNG Dose (μg/day) |
|---|---|
| <27.5 | 120 |
| ≥27.5 < 37.5 | 240 |
| ≥37.5 | 360 |

In related illustrative embodiments, the weight or BMI categories include the lower limits and exclude the upper limits, e.g., “>42.5≤47.5” is “≥42.5<47.5.” Additionally, in certain related illustrative embodiments of the invention, the doses per weight category can be +/−10% of the doses per weight category recited above.

In preferred embodiments of the invention, the dose of levonorgestrel is never less than about 90 μg/d, such that in the Constructive Products described above, the lower end of each range of doses of levonorgestrel is about 90 μg/d. For example:

Constructive Product 1a:

| Body Weight Category | LNG Dose (μg/day) |
|---|---|
| up to 120 pounds, | 90 to 120 |
| 120 to <150 pounds, | 104 to 210 |
| 150 to <180 pounds, | 154 to 312 |
| 180 to <210 pounds, | 179 to 364 |
| 210 to <240 pounds, | 156 to 317 |
| 240 to <270 pounds, | 226 to 457 |
| 270 and more pounds, | 208 to 422. |

Also, e.g.,

Constructive Product 10a:

| BMI Category | LNG Required Dose (μg/d) |
|---|---|
| ≤22.5 | 90-283 |
| >22.5 ≤ 27.5 | 90-335 |
| >27.5 ≤ 32.5 | 99-390 |
| >32.5 ≤ 37.5 | 127-447 |
| >37.5 ≤ 42.5 | 157-507 |
| >42.5 ≤ 47.5 | 187-569 |
| >47.5 ≤ 52.5 | 219-633. |

In other preferred embodiments of the invention, the dose of levonorgestrel is never less than a lowest dose of about 100 to about 120 μg/d, e.g., 100 μg/d, 110 μg/d, or 120 μg/d, such that in the Constructive Products described above, the lower end of each range of doses of levonorgestrel is 100, 110, or 120 μg/d. For example:

In other embodiments, variants of the above Constructive Products are employed in which the dose endpoints are rounded up or down to the nearest 5 μg/d or to the nearest 10 μg/d.

In general, as used in this specification and claims, “about” means +/−10%, except where such range would result in a nonsensical number such as an amount less than zero or when it is otherwise obvious from the context in which the word is used. Examples provided in the above description and examples are illustrative and not limiting.

The present invention is not limited to the embodiments described and exemplified above, but is capable of variation and modification within the scope of the appended claims and the above description including descriptions of illustrative embodiments or features thereof. Published literature, including but not limited to patent applications and patents, referenced in this specification are incorporated herein by reference as though fully set forth. The attached press release issued by Agile Therapeutics on Jan. 3, 2017 and entitled, “Agile Therapeutics Announces Positive Top-line Phase 3 Results,” and the attached poster by Nelson et al. and entitled, “The SECURE Study, a Real-World Trial of a Low-Dose Contraceptive Patch: Addressing the Changing U.S. Population” are also included in the present disclosure.

What we claim is:

1. A formulation comprising levonorgestrel delivering an amount of levonorgestrel that is based on the weight of women treated and that is within a range of amounts, with the maximum amounts being no higher than those amounts obtained using the equation
Maximum amount of LNG=−0.0084(woman's weight)²+5.1893(woman's weight)−375.79
and with the minimum amounts being no less than those amounts obtained using the equation
Minimum amount of LNG=−0.0041(woman's weight)²+2.5504(woman's weight)−184.36;
wherein amounts are expressed in micrograms per day (μg/d) and woman's weight is expressed in pounds;
or wherein the formulation also comprises a SHBG binding ligand, and the amount of LNG is adjusted to take into account the presence of the SHBG binding ligand.

2. A method of effecting contraception in a woman by internally administering to the woman levonorgestrel at a daily dose that is selected on the basis of her body weight wherein said daily dose of the progestin is within the range of Dₘᵢₙ and Dₘₐₓ wherein
Dₘᵢₙ=the lower of 90 or [(0.0041*X²)+(2.5504*X)−184.4];
Dₘₐₓ=[(0.0084*X²)+(5.1893*X)−375.8];
Dₘᵢₙ and Dₘₐₓ are minimum and maximum values in μg/d (+/−10%) of levonorgestrel or are contraceptively equivalent amounts of another progestin;
the progestin is coadministered with about 30 μg/d ethinyl estradiol or Dₘᵢₙ and Dₘₐₓ are adjusted if (A) (i) a different amount of ethinyl estradiol is coadministered, (ii) a different estrogen is coadministered or (iii) a non-estrogen SHBG binding ligand is coadministered and (B) the progestin is one that binds to SHBG;
X is the woman's body weight in pounds
or wherein the method delivers an amount of ethinyl estradiol that is greater or lesser than about 30 μg/d, the range including no ethinyl estradiol, and the amount of LNG is optionally adjusted to account for the presence of the ethinyl estradiol;
or wherein a SHBG binding ligand other than an estrogen, or an estrogen other than ethinyl estradiol, is delivered in an amount that is equivalent to about 30 μ/d ethinyl estradiol or to such greater or lesser amount, which includes no ethinyl estradiol.

3. A product line comprising a set of contraceptive products for women, wherein each set comprises one or more pharmaceutical dosage units for delivering a predetermined contraceptive amount of a progestin per day during a treatment period of at least 21 days;
the predetermined contraceptive amount is based on each woman's weight category;
each weight category is a range of between 5 pounds and 50 pounds;
the predetermined contraceptive amount of the progestin for each weight category is within the range of Dₘᵢₙ and Dₘₐₓ;
wherein
Dₘᵢₙ=the lower of 90 or [(0.0041*X²)+(2.5504*X)−184.4];
Dₘₐₓ=[(0.0084*X²)+(5.1893*X)−375.8];
Dₘᵢₙ and Dₘₐₓ are μg/d (+/−10%) of levonorgestrel or are contraceptively equivalent amounts of another progestin;
the progestin is coadministered with 30 μg/d ethinyl estradiol or Dₘᵢₙ and Dₘₐₓ are adjusted if (A) (i) a different amount of ethinyl estradiol is coadministered, (ii) a different estrogen is coadministered or (iii) a non-estrogen SHBG binding ligand is coadministered and (B) the progestin is one that binds to SHBG;
X is the woman's body weight in pounds
or wherein the method delivers an amount of ethinyl estradiol that is greater or lesser than about 30 μg/d (including no ethinyl estradiol) and the amount of LNG is optionally adjusted to take into account the ethinyl estradiol;
or wherein a SHBG binding ligand other than an estrogen, or an estrogen other than ethinyl estradiol, is delivered in an amount that is equivalent to about 30 μ/d ethinyl estradiol or to such greater or lesser amount (including no ethinyl estradiol).

4. A method of manufacturing a line of contraceptive pharmaceutical products for effecting contraception in a population of women of varying weight and/or BMI, said method comprising:
(A) analyzing the results of a clinical study of a progestin or progestin-estrogen contraceptive product in women of varying BMIs and/or body weights, said analysis including the steps of:
(i) preparing a graph of BMI or body weight versus number of pregnancies that occurred at each BMI or body weight;
(ii) selecting a minimum and a maximum acceptable pregnancy rate for all women and calculating Kd for the selected minimum and maximum acceptable pregnancy rates;
(iii) stratifying the women into subpopulations based on BMI or body weight range;
(iv) using the calculated Kd values for the minimum and maximum acceptable pregnancy rates, calculating the progestin dose required to achieve pregnancy rates within the selected minimum and maximum acceptable rates for all BMI or body weight subpopulations of women, using data from (i);
(B) manufacturing one set of contraceptive products of the same delivery type (e.g., oral, transdermal, implant, or IUD or other depot) for each weight/BMI category that delivers the required dose for each weight/BMI subpopulation of women.

5. The formulation, method or product line of any one of the preceding claims, wherein the required dose is adjusted to increase statistical confidence based on standard deviation and, optionally, wherein the dose is selected from within the range of the originally calculated required dose and the higher adjusted dose.

6. A system comprising one or more formulations, kits or methods, for personalized contraception in a woman, comprising the delivery of a contraceptively effective amount of a progestin, which amount is based on the body weight or BMI of the woman, and, optionally, an SHBG binding ligand.

7. The system of claim 6, adapted to deliver ethinyl estradiol at about 30 μg per day (or an equivalent amount of another estrogen or other SHBG binding ligand) and varying amounts of a progestin based on the body weight of the woman equivalent to the amount of levonorgestrel (LNG), selected from:
(a) for women weighing up to 120 pounds, an LNG equivalent amount of 90 μg per day or higher, optionally 90 to 150 μg per day;
(b) for women weighing between 120 and 150 pounds, an LNG equivalent amount of 100 μg per day or higher, optionally 100 to 240 μg per day;
(c) for women weighing between 150 and 180 pounds, an LNG equivalent amount of 140 μg per day or higher, optionally 140 to 350 μg/d;
(d) for women weighing between 180 and 210 pounds, an LNG equivalent amount of 150 μg per day or higher, optionally 170 to 400 μg/d;
(e) for women weighing between 210 and 240 pounds, an LNG equivalent amount of 150 μg per day or higher, optionally 170 to 400 μg/d;
(f) for women weighing between 240 and 270 pound, an LNG equivalent amount of 200 μg per day or higher, optionally 200 to 500 μg/d;
(g) for women of 270 pounds or more, an LNG equivalent amount of 200 μg per day or higher, optionally 200 to 500 μg/d
or wherein the formulations, kits, or methods deliver an amount of ethinyl estradiol that is greater or lesser than about 30 μg/d (including no ethinyl estradiol) and the amount of LNG (or LNG equivalent) is optionally adjusted to take into account the ethinyl estradiol;
or wherein a SHBG binding ligand other than an estrogen, or an estrogen other than ethinyl estradiol, is delivered in an amount that is equivalent to about 30 μ/d ethinyl estradiol or such greater or lesser amount (including no ethinyl estradiol).

8. The system of claim 6, adapted to deliver ethinyl estradiol at about 30 μg per day and varying amounts of a progestin based on the body weight of the woman equivalent to the amount of levonorgestrel (LNG) selected from:
(a) for women weighing up to 120 pounds, an LNG equivalent amount between 90 and 120 μg per day
(b) for women weighing between 120 and 150 pounds, an LNG equivalent amount between 105 and 215 μg per day
(c) for women weighing between 150 and 180 pounds, an LNG equivalent amount between 150 and 365 μg per day
(d) for women weighing between 180 and 210 pounds, an LNG equivalent amount between 150 and 365 μg per day
(e) for women weighing between 210 and 240 pounds, an LNG equivalent amount between 150 and 365 μg per day
(f) for women weighing between 240 and 270 pound, an LNG equivalent amount between 200 and 460 μg per day, and
(g) for women of 270 pounds or more, an LNG equivalent amount between 200 and 460 μg per day
or wherein the formulations, kits, or methods deliver an amount of ethinyl estradiol that is greater or lesser than about 30 μg/d (including no ethinyl estradiol) and the amount of LNG (or LNG equivalent) is optionally adjusted;
or wherein a SHBG binding ligand other than an estrogen, or an estrogen other than ethinyl estradiol, is delivered in an amount that is equivalent to about 30 μ/d ethinyl estradiol or such greater or lesser amount (including no ethinyl estradiol).

9. The system of claim 7 or claim 8, wherein the levonorgestrel equivalent amount provides a 95.5% confidence level that there will not be more than 3 pregnancies per 100 women years, wherein the levonorgestrel equivalent amounts are selected from:
(a) for women weighing up to 120 pounds, LNG equivalent amounts of between 90 and 215 μg per day
(b) for women weighing between 120 and 150 pounds, LNG equivalent amounts of 104 and 302 μg per day
(c) for women weighing between 150 and 180 pounds, LNG equivalent amounts of between 154 and 375 μg per day
(d) for women weighing between 180 and 210 pounds, LNG equivalent amounts of between 179 to 433 μg per day
(e) for women weighing between 210 and 240 pounds, LNG equivalent amounts of between 156 to 476 μg per day
(f) for women weighing between 240 and 270 pounds, LNG equivalent amounts of between 226 and 503 μg per day
(g) for women weighing 270 pounds or more, LNG equivalent amounts of between 208 to 516 μg per day;
or wherein the formulations, kits, or methods deliver an amount of ethinyl estradiol that is greater or lesser than about 30 μg/d (including no ethinyl estradiol) and the amount of LNG (or LNG equivalent) is optionally adjusted;
or wherein a SHBG binding ligand other than an estrogen, or an estrogen other than ethinyl estradiol, is delivered in an amount that is equivalent to about 30 μ/d ethinyl estradiol or such greater or lesser amount (including no ethinyl estradiol).

10. A method of effecting contraception in a woman comprising:
(a) determining the weight of the woman
(b) internally administering to the woman a progestin and an estrogen in accordance with the following schedule:
a progestin equivalent to 90 to 120 μg/d of levonorgestrel, and an estrogen equivalent to 30 μg/d of ethinyl estradiol if the woman weighs less than 130 pounds;
a progestin equivalent to 150 to 329 μg/d of levonorgestrel, and an estrogen equivalent to 30 μg/d of ethinyl estradiol if the woman weighs more than 130 pounds but less than 200 pounds; or
a progestin equivalent to 150-460 μg/d of levonorgestrel, and an estrogen equivalent to 30 μg/d of ethinyl estradiol if the woman weighs over 200 pounds.

11. A method of effecting contraception in a woman comprising:
(a) providing a contraceptive dosage form comprising a progestin;
(b) calculating based on clinical studies a dose of the progestin that is predicted to result in a pregnancy rate of 3% or less for each of a plurality of weight categories or BMI categories;
(c) determining the weight or BMI of the woman;
(d) administering to the woman the dosage form comprising the dose of the progestin that is predicted to result in a pregnancy rate of 3% or less for women in the woman's weight category or BMI category.

12. A method of effecting contraception in a woman having a body weight of 200 pounds or more, comprising administering to the woman:
(i) 340 mg/d LNG (or an equivalent amount of a different progestin) and 30 μg ethinyl estradiol (or an equivalent amount of another estrogen)
(ii) 260 mg/d LNG (or an equivalent amount of a different progestin) and 30 μg ethinyl estradiol (or an equivalent amount of another estrogen) or
(iii) 200 mg/d LNG (or an equivalent amount of a different progestin) and 30 μg ethinyl estradiol (or an equivalent amount of another estrogen).

13. The method of claim 12, wherein dose (i) is initially administered to the woman and if side effects develop then dose (ii) is administered instead and if side effects develop then dose (iii) is administered instead.

14. A method of effecting contraception in a woman having a body weight of 200 pounds or more, comprising administering to the woman:
(i) 420 mg/d LNG (or an equivalent amount of a different progestin) and 20 μg ethinyl estradiol (or an equivalent amount of another estrogen)
(ii) 330 mg/d LNG (or an equivalent amount of a different progestin) and 20 μg ethinyl estradiol (or an equivalent amount of another estrogen) or
(iii) 220 mg/d LNG (or an equivalent amount of a different progestin) and 20 μg ethinyl estradiol (or an equivalent amount of another estrogen).

15. The method of claim 14, wherein dose (i) is initially administered to the woman and if side effects develop then dose (ii) is administered instead and if side effects develop then dose (iii) is administered instead.

16. A method for effecting contraception in a woman by administering to the woman a pharmaceutical composition formulated to deliver ethinyl estradiol at about 30 μg per day (or an equivalent amount of another estrogen) and to deliver an amount of a progestin based on the potency of the progestin and on the body weight or the BMI of the woman,
wherein the amount of the progestin is equivalent to the amount of levonorgestrel (LNG) recited below,
(a) for women weighing <120 pounds, 90 to 120 μg LNG per day,
(b) for women weighing ≥120 <150 pounds, 100 to 210 μg LNG per day,
(c) for women weighing ≥150 and <180 pounds, 150 to 315 μg LNG per day,
(d) for women weighing ≥180 and <210 pounds, 150 to 364 μg LNG per day,
(e) for women weighing ≥210 and <240 pounds, 150 to 364 μg LNG per day,
(f) for women weighing ≥240 and <270 pound, 200 to 460 μg LNG per day, and
(g) for women weighing ≥270 pounds, 200 to 460 μg LNG per day,
optionally wherein the dose range endpoints per weight category recited above are +/−10% or +/−5%;
or wherein the method delivers an amount of ethinyl estradiol that is greater or lesser than about 30 μg/d (including no ethinyl estradiol) and the amount of LNG (or LNG equivalent) is optionally adjusted;
or wherein a SHBG binding ligand other than an estrogen, or an estrogen other than ethinyl estradiol, is delivered in an amount that is equivalent to about 30 μ/d ethinyl estradiol or to such greater or lesser amount (including no ethinyl estradiol).

17. The method of claim 16, wherein an amount at or near the high end of each dose range is administered to a woman based on her weight and if the woman experiences adverse effects associated with exogenous progestins, then reducing the amount of the progestin.

18. The method of claim 16 or claim 17, wherein the amount per day of the SHBG binding ligand is varied during a treatment cycle or the amount of the progestin is varied during a treatment cycle, or both.
